(19) United States
(12) Patent Application Publication
(10) Pub. No.: US 2025/0169874 A1
(43) Pub. Date: May 29, 2025

(54) UNIVERSAL SURGICAL FOOTSWITCH TOGGLING

(71) Applicant: Covidien LP, Mansfield, MA (US)

(72) Inventor: Henry D. Cook, Lyons, CO (US)

(73) Assignee: Covidien LP, Mansfield, MA (US)

(21) Appl. No.: 19/036,684

(22) Filed: Jan. 24, 2025

Related U.S. Application Data
(63) Continuation of application No. 17/354,036, filed on Jun. 22, 2021, now Pat. No. 12,226,143.
(60) Provisional application No. 63/042,163, filed on Jun. 22, 2020.

(51) Int. Cl. A61B 17/00; A61B 18/00; A61B 18/12

(52) U.S. Cl. CPC A61B 18/1206; A61B 2017/00115; A61B 2017/00973; A61B 2018/00607; A61B 2018/00839; A61B 2018/00928; A61B 2018/00994; A61B 2018/1253; A61B 2018/126

(57) ABSTRACT

An electrosurgical system includes an electrosurgical generator having a radio frequency source configured to operate in a plurality of modes and a controller configured to control the radio frequency source to output a radio frequency waveform corresponding to a mode from the plurality of modes. The system also includes a footswitch assembly coupled to the electrosurgical generator. The footswitch assembly includes a footswitch in communication with the controller. The footswitch is configured to output a footswitch activation signal to activate the radio frequency source. The footswitch assembly also includes a mode select button, which upon activation, is configured to instruct the controller to enter a mapping state during which the controller assigns an activation command for one mode from the plurality of modes to the footswitch.

### CROSS-REFERENCE TO RELATED APPLICATIONS

This application is a continuation of U.S. application Ser. No. 17/354,036 filed Jun. 22, 2021, which claims the benefit of U.S. Provisional Patent Application No. 63/042,163, filed on Jun. 22, 2020. The entire contents of the foregoing applications incorporated by reference herein. To the extent appropriate a claim of priority is made to each of the above disclosed applications.

### BACKGROUND

### Technical Field

The present disclosure relates to systems and methods for controlling an electrosurgical generator. In particular, the present disclosure relates to controlling a plurality of electrosurgical instrument functions using a footswitch by assigning any function activatable by the instrument to the footswitch.

### Background of Related Art

Electrosurgery involves application of high radio frequency electrical current to a surgical site to cut, ablate, desiccate, or coagulate tissue. In monopolar electrosurgery, a source or active electrode delivers radio frequency alternating current from the electrosurgical generator to the targeted tissue. A patient return electrode is placed remotely from the active electrode to conduct the current back to the generator.

In bipolar electrosurgery, return and active electrodes are placed in close proximity to each other such that an electrical circuit is formed between the two electrodes (e.g., in the case of an electrosurgical forceps). In this manner, the applied electrical current is limited to the body tissue positioned between the electrodes. Accordingly, bipolar electrosurgery generally involves the use of instruments where it is desired to achieve a focused delivery of electrosurgical energy between two electrodes.

Conventional electrosurgical generators utilize different footswitch assemblies for each functions (e.g., one footswitch controls a corresponding electrosurgical instrument coupled to a corresponding port of an electrosurgical generator). Thus, each instrument port also has a corresponding footswitch port to accommodate an individual footswitch. Each footswitch connector port ties the footswitch assembly to the instrument connector and each footswitch can only activate a single type of surgical energy through a surgical instrument. This configuration results in excessive number of footswitches, which may be confusing to the clinician and requires a large surface area for proper setup. Thus, there is a need for a simplified footswitch control system that can control multiple electrosurgical instruments and modalities using a single footswitch.

### SUMMARY

The present disclosure provides an electrosurgical system including an electrosurgical generator, a footswitch assembly, and one or more electrosurgical instruments. The footswitch includes a mode select button and one or more footswitches. When activated, the mode select button places the electrosurgical generator into a mapping state allowing for assigning an input from a button of the electrosurgical instrument or the generator to one of the footswitches. This allows for flexible use of the instruments, rather than using specific footswitches with only certain types of instruments.

According to one embodiment of the present disclosure, an electrosurgical system is disclosed. The system includes an electrosurgical generator having a radio frequency source configured to operate in a plurality of modes and a controller configured to control the radio frequency source to output a radio frequency waveform corresponding to a mode from the plurality of modes. The system also includes a footswitch assembly coupled to the electrosurgical generator. The footswitch assembly includes a footswitch in communication with the controller. The footswitch is configured to output a footswitch activation signal to activate the radio frequency source. The footswitch assembly also includes a mode select button, which upon activation, is configured to instruct the controller to enter a mapping state during which the controller assigns an activation command for one mode from the plurality of modes to the footswitch.

According to one aspect of the above embodiment, the electrosurgical system further includes an electrosurgical instrument configured to couple to the electrosurgical generator and to output the radio frequency waveform corresponding to the mode from the plurality of modes. The system may also include an ultrasonic instrument configured to couple to the electrosurgical generator and to convert the radio frequency waveform corresponding to the mode from the plurality of modes into vibrational energy. The electrosurgical instrument may further include an activation button configured to output an instrument activation signal to activate the radio frequency source, the instrument activation signal corresponding to the activation command for one mode from the plurality of modes. During the mapping state, the controller is further configured to receive the instrument activation signal to designate the activation command for assignment to the footswitch. The electrosurgical instrument may be a monopolar instrument and/or a bipolar instrument.

According to another aspect of the above embodiment, the footswitch assembly includes an indicator configured to display the mode corresponding to the activation command. The indicator may include a color-changing light and/or a display.

According to another embodiment of the present disclosure, an electrosurgical system is disclosed. The electrosurgical system includes an electrosurgical generator having a radio frequency source configured to operate in a plurality of modes; and a controller configured to control the radio frequency source to output a radio frequency waveform corresponding to a mode from the plurality of modes. The electrosurgical system also includes an electrosurgical instrument configured to couple to the electrosurgical generator and to output the radio frequency waveform corresponding to the mode from the plurality of modes. The electrosurgical instrument includes an activation button configured to output an instrument activation signal to activate the radio frequency source, the instrument activation signal corresponding to an activation command for one mode from the plurality of modes. The electrosurgical system further includes a footswitch assembly coupled to the electrosurgical generator. The footswitch assembly includes a footswitch in communication with the controller. The footswitch is configured to output a footswitch activation signal to activate the radio frequency source. The footswitch assembly further includes a mode select button, which upon activation, is configured to instruct the controller to enter a mapping state during which the controller assigns the activation command to the footswitch.

According to one aspect of the above embodiment, the electrosurgical instrument may be a monopolar instrument and/or a bipolar instrument.

According to another aspect of the above embodiment, the footswitch assembly may also include an indicator configured to display the mode corresponding to the activation command. The indicator includes a color-changing light and/or a display. The indicator may be disposed around a perimeter of the footswitch.

According to another embodiment of the present disclosure, a method for controlling an electrosurgical system is disclosed. The method includes coupling an electrosurgical instrument to an electrosurgical generator configured to operate in a plurality of modes and coupling a footswitch assembly to the electrosurgical generator. The method also includes activating a mode select button of the footswitch assembly to instruct a controller of the electrosurgical generator to enter a mapping state and activating an activation button of the electrosurgical instrument during the mapping state to output an instrument activation signal corresponding to an activation command for one mode from the plurality of modes. The method further includes assigning the activation command to a footswitch of the footswitch assembly.

According to one aspect of the above embodiment, the method includes indicating on an indicator of the footswitch assembly the mode corresponding to the activation command. Indicating may further include displaying a color indicative of the mode corresponding to the activation command. Indicating may further include displaying a name or an abbreviation of the mode corresponding to the activation command.

### BRIEF DESCRIPTION OF THE DRAWINGS

The present disclosure may be understood by reference to the accompanying drawings, when considered in conjunction with the subsequent, detailed description, in which:

FIG. 1 is a perspective view of an electrosurgical system according to an embodiment of the present disclosure;

FIG. 2 is a front view of a dual RF source electrosurgical generator of FIG. 1 according to an embodiment of the present disclosure;

FIG. 3 is a schematic diagram of the electrosurgical generator of FIG. 1 according to an embodiment of the present disclosure;

FIG. 4 is a perspective view of a footswitch according to one embodiment of the present disclosure;

FIG. 5 is a perspective view of a footswitch according to another embodiment of the present disclosure; and

FIG. 6 is a flow chart of a method for operating the electrosurgical generator of FIG. 1 according to an embodiment of the present disclosure.

### DETAILED DESCRIPTION

Embodiments of the presently disclosed electrosurgical system are described in detail with reference to the drawings, in which like reference numerals designate identical or corresponding elements in each of the several views. As used herein the term “distal” refers to the portion of the surgical instrument coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, an IOT device, a server system, or any programmable logic device.

In the following description, well-known functions or constructions are not described in detail to avoid obscuring the present disclosure in unnecessary detail. Those skilled in the art will understand that the present disclosure may be adapted for use with either an endoscopic instrument, a laparoscopic instrument, or an open instrument. It should also be appreciated that different electrical and mechanical connections and other considerations may apply to each particular type of instrument.

An electrosurgical generator according to the present disclosure may be used in monopolar and/or bipolar electrosurgical procedures, including, for example, cutting, coagulation, ablation, and vessel sealing procedures. The generator may include a plurality of outputs for interfacing with various ultrasonic and electrosurgical instruments (e.g., ultrasonic dissectors and hemostats, monopolar instruments, return electrode pads, bipolar electrosurgical forceps, footswitches, etc.). Further, the generator may include electronic circuitry configured to generate radio frequency energy specifically suited for powering ultrasonic instruments and electrosurgical devices operating in various electrosurgical modes (e.g., cut, blend, coagulate, division with hemostasis, fulgurate, spray, etc.) and procedures (e.g., monopolar, bipolar, vessel sealing).

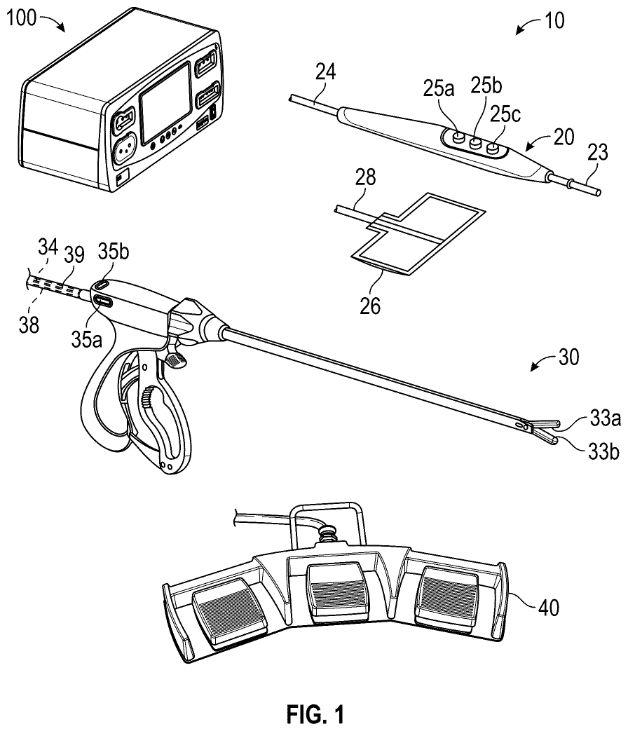

Referring to FIG. 1 an electrosurgical system 10 is shown which may include a plurality of electrosurgical instruments, such as a first instrument 20 and a second instrument 30. The first instrument 20 may be a monopolar electrosurgical instrument and the second instrument 30 may be a bipolar electrosurgical instrument. The first and second instruments 20 and 30 may be ultrasonic instruments having an ultrasonic transducer configured to vibrate a waveguide/end effector. The electrosurgical system 10 also includes a footswitch assembly 40 configured to control operation of either the first instrument 20 or the second instrument 30. In embodiments, the electrosurgical system 10 may operate with hybrid monopolar/bipolar electrosurgical instruments.

First instrument 20 include one or more active electrodes 23 (e.g., electrosurgical cutting probe, ablation electrode(s), etc.) for treating tissue of a patient. The system 10 may include a plurality of return electrode pads 26 that, in use, are disposed on a patient to minimize the chances of tissue damage by maximizing the overall contact area with the patient. Electrosurgical alternating RF current is supplied to the first instrument 20 by a generator 100 via supply line 24. The alternating RF current is returned to the generator 100 through the return electrode pad 26 via a return line 28. In addition, the generator 100 and the return electrode pads 26 may be configured for monitoring tissue-to-patient contact to ensure that sufficient contact exists therebetween. The first instrument 20 includes a plurality of activation buttons 25a, 25b, 25c, each of which is configured to activate the electrosurgical generator 100 to operate in a corresponding mode and output an electrosurgical waveform for treating tissue.

Second instrument 30 is shown as forceps. In embodiments, the second instrument 30 may be tweezers. The second instrument 30 includes a pair of electrodes 33a and 33b for treating tissue of a patient, which are connected to the electrosurgical generator 100 through a cable 39 that includes the supply and return lines 34, 38. The second instrument 30 is coupled to the electrosurgical generator 100 at a port having connections to the active and return terminals (e.g., pins) via a plug (not shown) disposed at the end of the cable 39, wherein the plug includes contacts from the supply and return lines 34, 38 as described in more detail below. The second instrument 30 includes a plurality of activation buttons 35a and 35b each of which is configured to activate the electrosurgical generator 100 to operate in a corresponding mode and output an electrosurgical waveform for treating tissue.

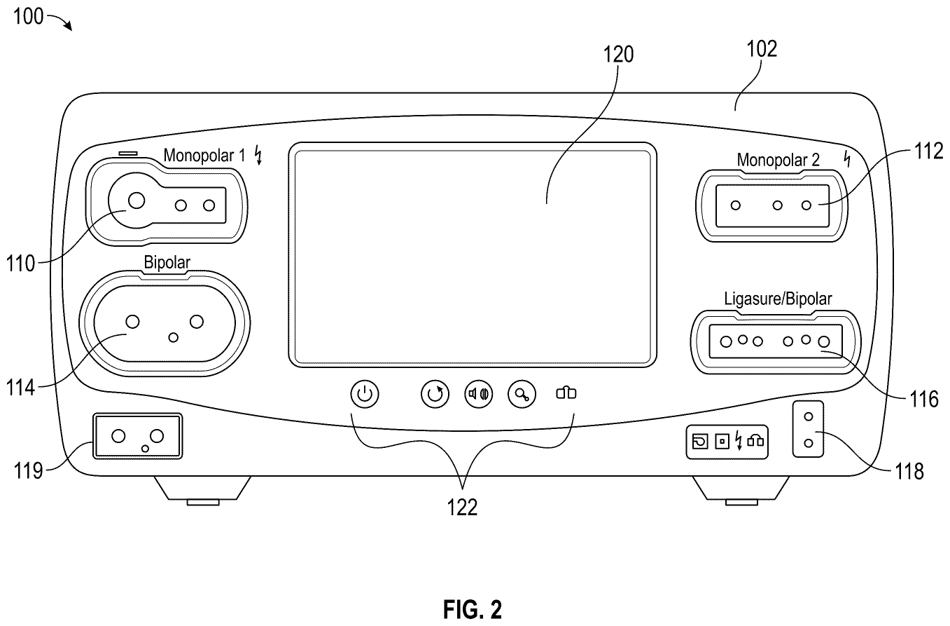

With reference to FIG. 2, a front face 102 of the generator 100 is shown. The generator 100 may include a plurality of ports 110, 112, 114, 116 to accommodate various types of electrosurgical instruments and a port 118 for coupling to the return electrode pad 26 and a port 119 configured to couple to the footswitch assembly 40. The ports 110 and 112 are configured to couple to the first instrument 20. The ports 114 and 116 are configured to couple to the bipolar electrosurgical instruments 30. The generator 100 includes a display 120 for providing the user with variety of output information (e.g., intensity settings, treatment complete indicators, etc.). The display 120 is a touchscreen configured to display a menu corresponding to each of the ports 110, 112, 114, 116 and the instrument coupled. The user also adjusts inputs by touching corresponding menu options. The generator 100 also includes suitable input controls 122 (e.g., buttons, activators, switches, touch screen, etc.) for controlling the generator 100.

The generator 100 is configured to operate in a variety of modes and is configured to output monopolar and/or bipolar waveforms corresponding to the selected mode. Each of the modes may be activated by the buttons 25a, 25b, 25c of the first instrument 20 and the buttons 35a and 35b of the second instrument 30 and/or the footswitch assembly 40. Each of the modes operates based on a preprogrammed power curve that limits how much power is output by the generator 100 at varying impedance ranges of the load (e.g., tissue). Each of the power curves includes power, voltage and current control ranges that are defined by the user-selected intensity setting and the measured minimum impedance of the load.

The generator 100 may operate in the following monopolar modes, which include, but are not limited to, cut, blend, division with hemostasis, fulgurate and spray. The generator 100 may operate in the following bipolar modes, including bipolar cutting, bipolar coagulation, automatic bipolar which operates in response to sensing tissue contact, and various algorithm-controlled vessel scaling modes.

Each of the RF waveforms may be either monopolar or bipolar RF waveforms, each of which may be continuous or discontinuous and may have a carrier frequency from about 200 kHz to about 500 kHz. As used herein, continuous waveforms are waveforms that have a 100% duty cycle. In embodiments, continuous waveforms are used to impart a cutting effect on tissue. Conversely, discontinuous waveforms are waveforms that have a non-continuous duty cycle, e.g., below 100%. In embodiments, discontinuous waveforms are used to provide coagulation effects to tissue.

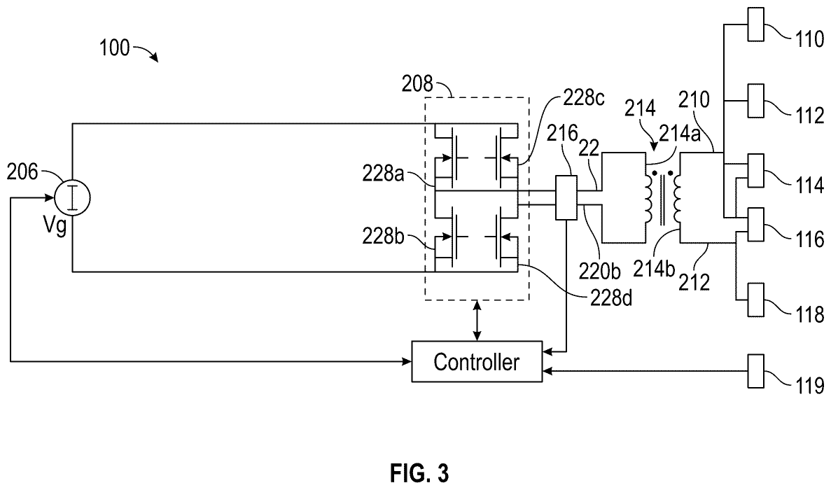

With reference to FIG. 3, the generator 100 includes a controller 204, a power supply 206, and a RF inverter 208. The power supply 206 may be high voltage, DC power supplies connected to a common AC source (e.g., line voltage) and provide high voltage, DC power to their respective RF inverter 208, which then convert DC power into a RF waveform through active terminal 210 and return terminal 212 corresponding to the selected mode.

The active terminal 210 and the return terminal 212 are coupled to the RF inverter 208 through an isolation transformer 214. The isolation transformer 214 includes a primary winding 214a coupled to the RF inverter 208 and a secondary winding 214b coupled to the active and return terminals 210 and 212.

Electrosurgical energy for energizing the first instrument 20 is delivered through the ports 110 and 112, each of which is coupled to the active terminal 210. RF energy is returned through the return electrode pad 26 coupled to the port 118, which in turn, is coupled to the return terminal 212. The secondary winding 214b of the isolation transformer 214 is coupled to the active and return terminals 210 and 212. RF energy for energizing the second instrument 30′ is delivered through the ports 114 and 116, each of which is coupled to the active terminal 210 and the return terminal 212. The generator 100 may include a plurality of steering relays or other switching devices configured to couple the active terminal 210 and the return terminals 212 to various ports 110, 112, 114, 116, 118 based on the combination of the monopolar and bipolar electrosurgical instruments 20 and 30 being used.

The RF inverter 208 is configured to operate in a plurality of modes, during which the generator 100 outputs corresponding waveforms having specific duty cycles, peak voltages, crest factors, etc. It is envisioned that in other embodiments, the generator 100 may be based on other types of suitable power supply topologies. RF inverter 208 may be a resonant RF amplifier or non-resonant RF amplifier, as shown. A non-resonant RF amplifier, as used herein, denotes an amplifier lacking any tuning components, i.e., conductors, capacitors, etc., disposed between the RF inverter and the load, e.g., tissue.

The controller 204 may include a processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to perform the calculations and/or set of instructions described herein.

The controller 204 is operably connected to the power supply 206 and/or RF inverter 208 allowing the processor to control the output of the RF source 202 of the generator 100 according to either open and/or closed control loop schemes. A closed loop control scheme is a feedback control loop, in which a plurality of sensors measures a variety of tissue and energy properties (e.g., tissue impedance, tissue temperature, output power, current and/or voltage, etc.), and provide feedback to the controller 204. The controller 204 then controls the power supply 206 and/or RF inverter 208, which adjust the DC and/or RF waveform, respectively.

The generator 100 according to the present disclosure may also include a plurality of sensors 216, each of which monitors output of the RF source 202 of the generator 100. The sensor 216 may be any suitable voltage, current, power, and impedance sensors. The sensors 216 are coupled to leads 220a and 220b of the RF inverter 208. The leads 220a and 220b couple the RF inverter 208 to the primary winding 214a of the transformer 214. Thus, the sensors 216 are configured to sense voltage, current, and other electrical properties of energy supplied to the active terminal 210 and the return terminal 212.

In further embodiments, the sensor 216 may be coupled to the power supply 206 and may be configured to sense properties of DC current supplied to the RF inverter 208. The controller 204 also receives input (e.g., activation) signals from the display 120, the input controls 122 of the generator 100 and/or the instruments 20 and 30 (e.g., buttons 25a, 25b, 25c, 35a, 35b), and the footswitch assembly 40. The controller 204 adjust power outputted by the generator 100 and/or perform other control functions thereon in response to the input signals.

The RF inverter 208 includes a plurality of switching elements 228a-228d, which are arranged in an H-bridge topology. In embodiments, RF inverter 208 may be configured according to any suitable topology including, but not limited to, half-bridge, full-bridge, push-pull, and the like. Suitable switching elements include voltage-controlled devices such as transistors, field-effect transistors (FETs), combinations thereof, and the like. In embodiments, the FETs may be formed from gallium nitride, aluminum nitride, boron nitride, silicon carbide, or any other suitable wide bandgap materials.

The controller 204 is in communication with the RF inverters 208, and in particular, with the switching elements 228a-228d. Controller 204 is configured to output control signals, which may be pulse-width modulated (“PWM”) signals, to switching elements 228a-228d. In particular, controller 204 is configured to modulate a control signal supplied to switching elements 228a-228d of the RF inverter 208. The control signal provides PWM signals that operate the RF inverter 208 at a selected carrier frequency. Additionally, controller 204 are configured to calculate power characteristics of output of the RF source 202 of the generator 100, and control the output of the generator 100 based at least in part on the measured power characteristics including, but not limited to, voltage, current, and power at the output of RF inverters 208.

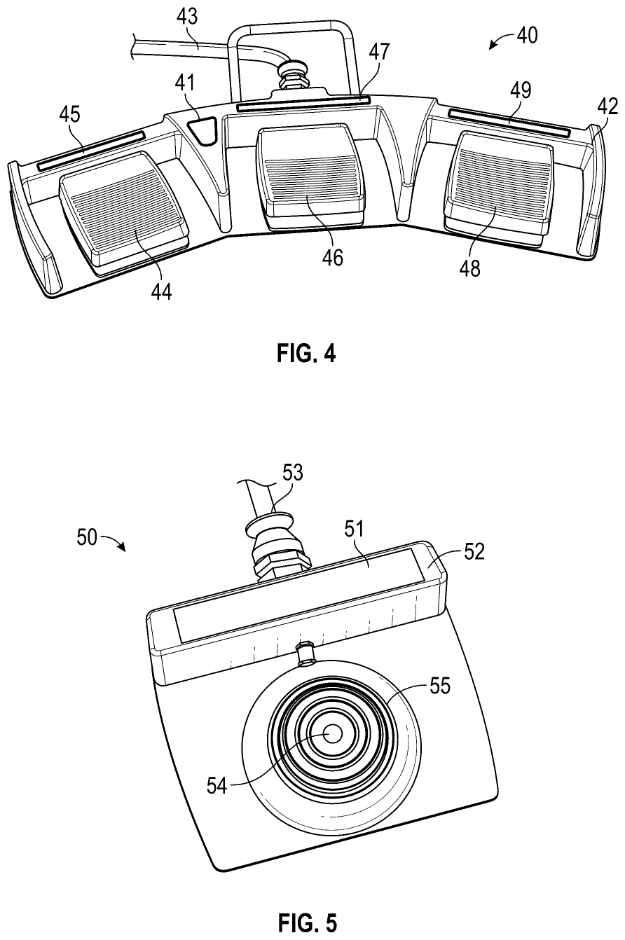

With reference to FIG. 4, the footswitch assembly 40 includes a base housing 42 accommodating a plurality of footswitches 44, 46, 48, which are shown as pedal switches. The footswitches 44, 46, 48 may be any mechanically actuated electrical switch such as a toggle switch, a pushbutton switch, which requires continuous engagement, and combinations thereof (e.g., a pushbutton latching switch or a pushbutton continuous switch). The footswitches 44, 46, 48 may be on-off switches, latching switches, continuous switches, and the like. As used herein a latching switch maintains the state in which the switch was placed following last engagement. When activated, the footswitches 44, 46, 48 output activation signals to the controller 204, which then activates a selected mode and output an electrosurgical waveform.

The footswitch assembly 40 is coupled to the port 119 of the generator via a cable 43 and is coupled to the controller 204. This allows the footswitch assembly 40 to transmit activation signals to the controller 204 and for the controller 204 to reconfigure operation of the footswitch assembly 40. The footswitch assembly 40 is configured to control any of the instruments coupled to any of the ports 110, 112, 114, 116 of the generator 100. This is accomplished by configuring operation of the footswitch assembly 40. Configuration of the footswitch assembly 40 may be done at any point during use of the generator 100. The footswitch assembly 40 includes a mode select button 41, which allows for assigning any one of the footswitches 44, 46, 48 to a particular button function of the instrument 20 or 30. In embodiments, the footswitches 44, 46, 48 may be mapped to any one of the functions or modes that are activated by the buttons 25a, 25b, 25c of the monopolar instrument 20 or the buttons 35a and 35b of the bipolar instrument 30. Thus, the footswitches 44 and 46 may be mapped to the buttons 25a and 25b while the footswitch 48 may be mapped to the button 35a. In further embodiments, the footswitches 44, 46, 48 may be mapped to any of the modes or functions that are activated using the interface of the generator 100, namely, through the display 120 and the input controls 122.

Mapping of the modes may be accomplished by initially pressing the mode select button 41. This instructs the controller 204 to enter a mapping state during which the controller 204 is configured to receive an activation signal from the instrument 20, 30, or the generator 100 and maps an activation command corresponding to the activation signal to one of the footswitches 44, 46, 48. Thus, the controller 204 waits for engagement of one of the buttons 25a, 25b, 25c of the monopolar instrument 20, the buttons 35a and 35b of the bipolar instrument 30, or on the display 120 and the input controls 122 of the generator 100. After the mode is selected, the user then engages one of the footswitches 44, 46, 48 and the mode activation command is mapped to the engaged footswitch 44, 46, 48. In embodiments, the mode select button 41 may be pressed continuously while the mapping process is performed, this indicates to the controller 204 to disengage any RF power application and to map the selected function to the footswitch assembly 40.

During mapping of functions to the footswitch assembly 40, the generator 100 may display the order of steps to map the functions on the display 120, such that the user is guided through the process and to further indicate that the function has been successfully mapped. In embodiments, the order of selecting the function to be mapped and selecting which of the footswitches 44, 46, 48 is being mapped may be reversed, such that the user initially selects the footswitch 44, 46, 48 followed by selection of the function. Once the function is mapped to one of the footswitches 44, 46, 48 the user may then continue to activate that function by pressing the footswitch 44, 46, 48. The assigned function of the footswitch 44, 46, 48 may be overwritten at any time by pressing the mode select button 41 and repeating the assignment process.

The footswitch assembly 40 also includes indicators 45, 47, 49 corresponding to each of the footswitches 44, 46, 48, respectively. Each of the indicators 45, 47, 49 is configured to indicate to the user the currently assigned function. The indicators 45, 47, 49 may be displays configured to display the currently assigned function or instrument using text (e.g., name, abbreviation and/or numbers). The displayed information may also be color-coded. In further embodiments, the indicators 45, 47, 49 may be color-changing lights (e.g., full-spectrum LEDs) configured to change color to a predefined color associated with the function or port 110, 112, 114, 116 of the generator 100. The assigned color of each of the ports 110, 112, 114, 116 may be shown on the display 120 such that the assigned color corresponds to the color on the graphical user interface of the display 120. The indicators 45, 47, 49 may be of any suitable shape or size that is visible by the user.

Another embodiment of the footswitch assembly 50 is shown in FIG. 5. The footswitch assembly 50 has the same components and performs the same function as the footswitch assembly 40, in which like reference numerals designate identical or corresponding elements, however, the differences are described below. The footswitch assembly 50 includes a base housing 52 having a single footswitch 54, a cable 53 coupling the footswitch assembly 50 to the generator 100, a mode select button 51, and an indicator 55. The footswitch assembly 50 is substantially similar to the footswitch assembly 40 except that the footswitch assembly 50 includes only one footswitch 54, which is shown as a pushbutton switch. In addition, the indicator 55 is shown as a lighted outline (e.g., a light ring) disposed around the footswitch 54 and is configured to change color based on the selected function, which is accomplished in the manner described above with respect to the footswitch assembly 40. In embodiments, the indicator 55 may have any suitable shape conforming to the outline of the footswitch 54, e.g., a rectangular footswitch 54 may have a rectangular outline along the perimeter of the footswitch 54. In further embodiments, the indicator 55 may be a display that is also shaped to conform to the shape of the footswitch 54. The generator 100 may include a plurality of ports 119 such that any number of footswitch assemblies 40 or 50 may be used. In further embodiments, footswitch assemblies may include any number of footswitches.

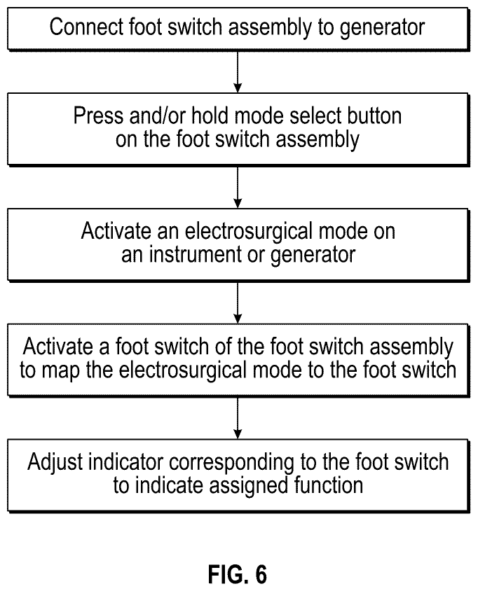

With reference to FIG. 6, a method of configuring and using the footswitch assembly 40 is described. The footswitch assembly 50 may be used the same method. Initially, the footswitch assembly 40 is connected to the port 119 of the generator 100. The controller 204 may be configured to detect that the footswitch assembly 40 is properly connected to the port 119 and/or the footswitch assembly 40 is an approved accessory. In addition, one or more of the first instrument 20 and/or the second instrument 30 are also coupled to the generator 100 through one of the ports 110, 112, 114, 116. The user may also configure the generator 100 to operate in a corresponding electrosurgical mode by modifying settings (e.g., intensity).

Thereafter, the user engages the mode select button 41 of the footswitch assembly 40 to commence function assignment to one of the footswitches 44, 46, 48. The mode select button 41 may be pressed and/or held during assignment. Thereafter, the user activates the electrosurgical mode by engaging of one of the buttons 25a, 25b, 25c of the monopolar instrument 20 or the buttons 35a and 35b of the bipolar instrument 30 or on the display 120 and the input controls 122 of the generator 100.

After the mode is selected, the user then engages one of the footswitches 44, 46, 48 to select to which of the footswitches 44, 46, 48 the mode activation command is mapped. In embodiments, the mode select button 41 may be pressed continuously while the mapping process is performed, this indicates to the controller 204 to disengage any RF power application and to map the selected function to the footswitch assembly 40.

In embodiments, the order of selecting the function to be mapped and selecting which of the footswitches 44, 46, 48 the function is mapped to may be reversed, such that the user initially selects the footswitch 44, 46, 48 followed by selection of the function. The controller 204 maps the function to the selected footswitch 44, 46, 48 and also adjusts the corresponding indicator 45, 47, 49 to display the assigned function. Thereafter, the user may activate the assigned function by engaging one of the footswitches 44, 46, 48.

While several embodiments of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope of the claims appended hereto.

What is claimed is:

1. An electrosurgical system, comprising:
an electrosurgical instrument operable in a plurality of modes;
a footswitch assembly comprising a plurality of footswitches configured to activate the plurality of modes, the plurality of footswitches associated with a plurality of activation signals, respectively, wherein the footswitch assembly outputs an activation signal of the plurality of activation signals based on activation of one of the plurality of footswitches; and
an electrosurgical generator comprising:
a footswitch interface for receiving the activation signal from the footswitch assembly;
memory storing a mapping between the plurality of activation signals and the plurality of modes, wherein the electrosurgical generator is operable in a mapping mode during which the mapping is established;
a radiofrequency source configured to generate a radiofrequency waveform based on a mode mapped to the activation signal; and
one or more instrument ports coupling the electrosurgical instrument to the electrosurgical generator and transmitting the radiofrequency waveform to the electrosurgical instrument, the electrosurgical instrument being one of a plurality of instrument types accepted by at least one of the one or more instrument ports.

2. The electrosurgical system of claim 1, wherein the footswitch interface includes a wireless receiver, and the footswitch assembly includes a corresponding wireless transmitter.

3. The electrosurgical system of claim 1, wherein the footswitch interface includes a footswitch port configured to receive a connector of the footswitch assembly or a wireless communication receiver.

4. The electrosurgical system of claim 1, wherein at least one of the electrosurgical instrument, the footswitch assembly, or the electrosurgical generator comprises a user input element that causes the electrosurgical generator to be placed in the mapping mode when activated.

5. The electrosurgical system of claim 1, wherein the footswitch assembly is mappable to any electrosurgical instrument coupled to any of the one or more instrument ports.

6. The electrosurgical system of claim 1, wherein the one or more instrument ports includes at least one monopolar instrument port and at least one bipolar instrument port.

7. An electrosurgical system, comprising:
an electrosurgical instrument comprising a plurality of user input elements configured to activate a plurality of operational modes;
a footswitch assembly comprising a plurality of footswitches configured to activate the plurality of operational modes, the plurality of footswitches associated with a plurality of activation signals, respectively; and
an electrosurgical generator comprising:
memory storing a mapping between the plurality of activation signals and the plurality of operational modes, wherein the electrosurgical generator is operable in a mapping mode during which the mapping is established;
a footswitch interface configured to receive an activation signal of the plurality of activation signals from the footswitch assembly, the activation signal corresponding to an operational mode as defined by the mapping;
a radiofrequency source configured to generate a radiofrequency waveform based on the operational mode; and
one or more instrument ports configured to transmit the radiofrequency waveform to the electrosurgical instrument, the electrosurgical instrument being one of a plurality of instrument types compatible with at least one of the one or more instrument ports.

8. The electrosurgical system of claim 7, wherein the plurality of user input elements includes a plurality of buttons.

9. The electrosurgical system of claim 8, wherein a first user input element of the plurality of user input elements corresponds to a first footswitch of the plurality of footswitches, and activation of either the first user input element or the first footswitch activates a first operational mode of the electrosurgical generator.

10. The electrosurgical system of claim 7, wherein at least one of the electrosurgical instrument, the footswitch assembly, or the electrosurgical generator comprises a user input element that causes the electrosurgical generator to be placed in the mapping mode when activated.

11. The electrosurgical system of claim 7, wherein the footswitch assembly is mappable to any electrosurgical instrument coupled to any of the one or more instrument ports.

12. The electrosurgical system of claim 7, wherein the one or more instrument ports includes at least one monopolar instrument port and at least one bipolar instrument port.

13. The electrosurgical system of claim 7, wherein the footswitch interface includes a wireless receiver, and the footswitch assembly includes a corresponding wireless transmitter.

14. The electrosurgical system of claim 7, wherein the footswitch interface includes a footswitch port configured to receive a connector of the footswitch assembly or a wireless communication receiver.

15. An electrosurgical system, comprising:
a footswitch assembly comprising a plurality of footswitches associated with a plurality of activation signals, respectively, the footswitch assembly configured to output an activation signal of the plurality of activation signals based on an activated footswitch of the plurality of footswitches; and
an electrosurgical generator operable in a mapping mode and a plurality of radiofrequency modes, the electrosurgical generator comprising:
a footswitch interface for receiving the activation signal, the activation signal corresponding to a radiofrequency mode of the plurality of radiofrequency modes based on a mapping between the plurality of activation signals and the plurality of radiofrequency modes, wherein the mapping is established when the electrosurgical generator is in the mapping mode;
a radiofrequency source configured to generate a radiofrequency waveform based on the radiofrequency mode; and
one or more instrument ports for coupling an electrosurgical instrument to the electrosurgical generator and transmitting the radiofrequency waveform to the electrosurgical instrument, the one or more instrument ports compatible with a plurality of electrosurgical instrument types.

16. The electrosurgical system of claim 15, wherein the footswitch interface includes a wireless receiver, and the footswitch assembly includes a corresponding wireless transmitter.

17. The electrosurgical system of claim 15, wherein the footswitch interface includes a footswitch port configured to receive a connector of the footswitch assembly or a wireless communication receiver.

18. The electrosurgical system of claim 15, wherein at least one of the electrosurgical instrument, the footswitch assembly, or the electrosurgical generator comprises a user input element that causes the electrosurgical generator to be placed in the mapping mode when activated.

19. The electrosurgical system of claim 15, wherein the footswitch assembly is mappable to any electrosurgical instrument coupled to any of the one or more instrument ports.

20. The electrosurgical system of claim 15, wherein the one or more instrument ports includes at least one monopolar instrument port and at least one bipolar instrument port.
